(19) United States
(12) Patent Application Publication
(10) Pub. No.: US 2021/0267816 A1
(43) Pub. Date: Sep. 2, 2021

(54) Absorbent Article

(71) Applicant: 3M INNOVATIVE PROPERTIES COMPANY, St. Paul, MN (US)

(72) Inventor: Hiroshi Sakurai, Kanagawa (JP)

(21) Appl. No.: 17/250,223

(22) PCT Filed: Jun. 26, 2019
(86) PCT No.: PCT/IB2019/055415

§ 371 Date: Dec. 18, 2020

(30) Foreign Application Priority Data
Jun. 29, 2018 (JP) 2018-25138

(51) Int. Cl. A61F 13/494; A61F 13/53

(52) U.S. Cl. CPC A61F 13/4942; A61F 2013/49074; A61F 13/53

(57) ABSTRACT

The absorbent article is an absorbent article covering a crotch portion of a wearer from an abdominal side to a back side with a sheet-like main body, the absorbent article including: the main body including an abdominal side edge, a back side edge, pair of crotch side edges extending between the abdominal side edge and the back side edge, and an absorbent region in which an absorbent is disposed; a pair of restraint portions provided on the main body along the pair of crotch side edges, and configured to suppress leakage of a target object from the absorbent region; and a bonding member in a strip shape provided to the main body on a side of at least one of the abdominal side edge and the back side edge, wherein the bonding member is provided extending between the pair of restraint portions, and both ends of the bonding member are disposed between the restraint portions and the main body at one end side of the restraint portion in a longitudinal direction.

### TECHNICAL FIELD

The present invention relates to an absorbent article.

### BACKGROUND ART

In the related art, an article described in JP H11-305781A is known as an absorbent article. JP H11-305781A describes an absorbent article covering a crotch portion of a wearer from an abdominal side to a back side with a sheet-like main body. The absorbent article includes a pair of restraint members that are provided on the main body along a pair of crotch side edges, and suppress leakage of a target object such as urine and feces from an absorbent region.

### SUMMARY OF THE INVENTION

There has been a demand for an absorbent article to further improve a function of suppressing the leakage of the target object from the main body. In other words, there is a demand to provide an absorbent article that can improve the function of suppressing the leakage. According to the present invention, an absorbent article capable of improving the function of suppressing the leakage can be provided.

An absorbent article according to an aspect of the present invention is an absorbent article covering a crotch portion of a wearer from an abdominal side to a back side with a sheet-like main body, the absorbent article including: the main body including an abdominal side edge, a back side edge, a pair of crotch side edges extending between the abdominal side edge and the back side edge, and an absorbent region in which an absorbent is disposed; a pair of restraint portions provided on the main body along the pair of crotch side edges, and configured to suppress leakage of a target object from the absorbent region; and a bonding member in a strip shape provided to the main body on a side of at least one of the abdominal side edge and the back side edge, wherein the bonding member is provided extending between the pair of restraint portions, and both ends of the bonding member are disposed between the restraint portions and the main body at one end side of the restraint portion in a longitudinal direction.

### BRIEF DESCRIPTION OF THE DRAWINGS

FIG. 1 is a perspective view illustrating a diaper that is an absorbent article according to the present embodiment.

FIG. 2 is a schematic plan view of a diaper.

FIG. 3A is an enlarged cross-sectional view taken along a line IIIa-IIIa illustrated in FIG. 2, and FIG. 3B is a cross-sectional view taken along a line IIIb-IIIb illustrated in FIG. 2.

FIG. 4 is a cross-sectional view taken along a line IV-IV illustrated in FIG. 2.

FIGS. 5A and 5B are enlarged cross-sectional views illustrating a layer configuration of a bonding member.

FIGS. 6A and 6B are cross-sectional views illustrating a diaper according to a modification example, and is a cross-sectional view corresponding to FIGS. 3A and 3B.

### DETAILED DESCRIPTION

An embodiment according to the present invention is described in detail below with reference to the accompanying drawings. Note that, in the following description, the same or equivalent elements are designated by the same reference signs, and the duplicated description is omitted.

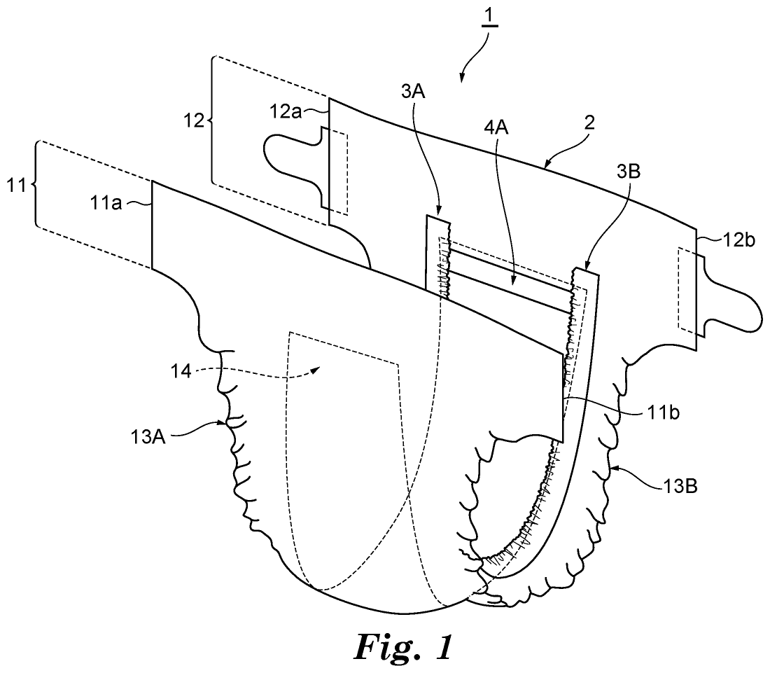

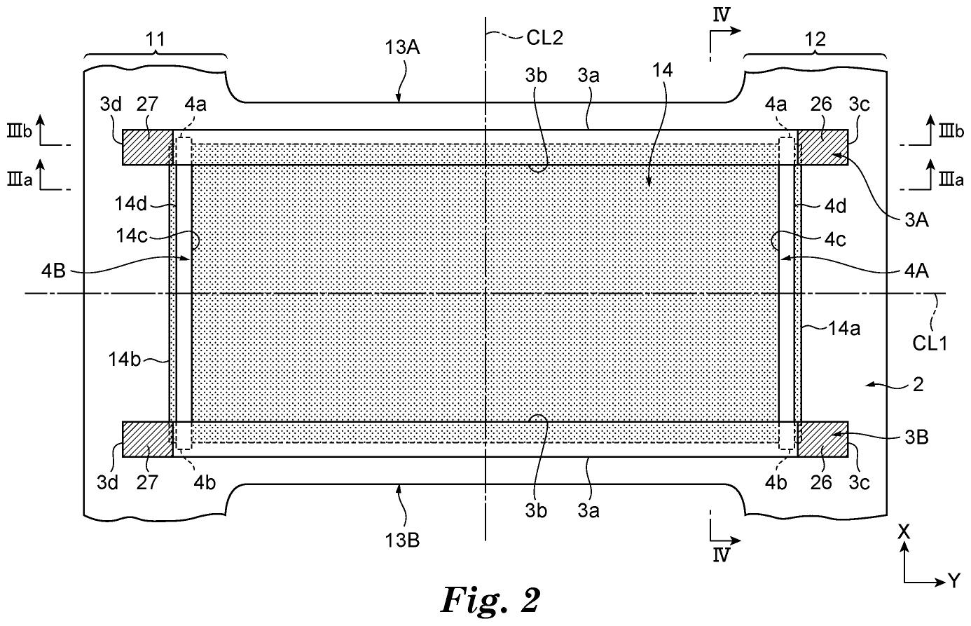

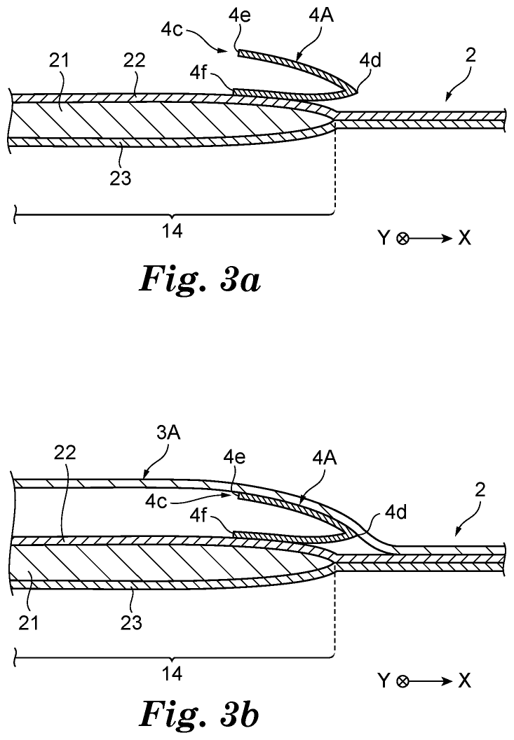

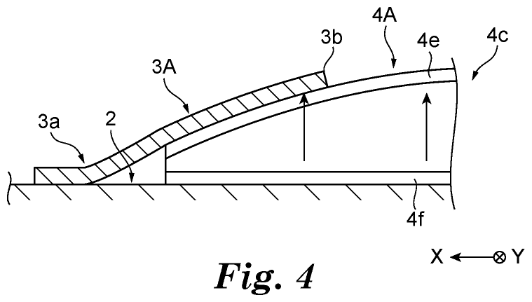

FIG. 1 is a perspective view illustrating a diaper 1 that is an absorbent article according to the present embodiment. FIG. 2 is a schematic plan view of the diaper 1. FIG. 3A is an enlarged cross-sectional view taken along a line illustrated in FIG. 2, and FIG. 3B is a cross-sectional view taken along a line IIIb-IIIb illustrated in FIG. 2. FIG. 4 is a cross-sectional view taken along a line IV-IV illustrated in FIG. 2.

The diaper 1 is a member that covers a crotch portion of a wearer from an abdominal side to a back side with a sheet-like main body 2. This allows the diaper 1 to absorb a target object discharged from the crotch portion of the wearer and prevent leakage of the target object. Note that the target object of the diaper 1 is a moisture, a solid, and a semi-solid, and examples thereof include urine and feces. As illustrated in FIGS. 1 to 4, the diaper 1 includes the main body 2, restraint portions 3A and 3B, and bonding members 4A and 4B.

As illustrated in FIGS. 3A and 3B, the main body 2 includes a backsheet 23, a liquid-permeable topsheet 22 laminated inside the backsheet 23, and an absorbent layer 21 including a liquid-absorptive polymeric absorbent enclosed between the backsheet 23 and the topsheet 22. The backsheet 23 is generally formed of a laminate of a liquid-impermeable film and a non-woven layer, in which the liquid-impermeable film is on the absorbent layer 21 side and the non-woven layer is on an outer surface side of a body of the diaper 1. Specifically, when the diaper 1 is worn, the topsheet 22 is located such that an inner surface of the body of the diaper 1 is adjacent to or closer to a wearer's skin, and the backsheet 23 (the non-woven layer thereof) is located such that the outer surface of the body of the diaper 1 is adjacent to or closer to a wearer's clothing or the like. Here, examples of the non-woven layer used in the backsheet 23 include a non-woven fabric which is produced by spunbonding or the like and made of polyolefin, PET, or the like having a mass in a range from 15 to 40 g/m², preferably, from 25 to 35 g/m², and a fiber density in a range from 0.06 to 0.1 g/cm³, preferably, from 0.07 to 0.09 g/cm³. In this case, the non-woven layer can be used to fix the non-woven layer with a connecting portion.

As illustrated in FIGS. 1 and 2, the main body 2 includes an abdominal side edge 11, a back side edge 12, a pair of crotch side edges 13A and 13B, and an absorbent region 14. The abdominal side edge 11 is a strip-shaped portion located on an abdominal side of the wearer. The back side edge 12 is a strip-shaped portion located on the back side of the wearer. Since the diaper 1 of the present embodiment is an open-type diaper, the abdominal side edge 11 and the back side edge 12 are separated from each other. The back side edge 12 includes fastener members 16A and 16B respectively provided to ends 12a and 12b on lateral sides of the back side edge. The fastener member 16A is bonded to a region near an end 11a on a lateral side of the abdominal side edge 11. The fastener member 16B is bonded to a region near an end 11b on a lateral side of the abdominal side edge 11.

When the diaper 1 is worn by the wearer, the abdominal side edge 11 is wound on a portion on the abdominal side of the wearer, the back side edge 12 is wound on a portion on the back side of the wearer, and the abdominal side edge 11 and the back side edge 12 are fixed via the fastener members 16A and 16B. The crotch side edges 13A and 13B are the edges extending between the abdominal side edge 11 and the back side edge 12. The crotch side edges 13A and 13B are located near legs near a wearer's crotch. The crotch side edges 13A and 13B are manufactured to be stretchable.

Note that, in the following description, a description may be made with XY axes being set with respect to the main body 2 in an open state as illustrated in FIG. 2. An X-axis direction is a direction in which the crotch side edges 13A and 13B are opposite to each other, the crotch side edge 13A side is a positive side, and the crotch side edge 13B is a negative side. A Y-axis direction is a direction in which the abdominal side edge 11 and the back side edge 12 are opposite to each other, the back side edge 12 side is a positive side, and the abdominal side edge 11 side is a negative side. A center line CL1 of the main body 2 in the X-axis direction and a center line CL2 of the main body 2 in the Y-axis direction are set.

The absorbent region 14 is a region in which an absorbent that absorbs a non-solid component in the target object is disposed. The absorbent region 14 is provided at a position surrounded by the abdominal side edge 11, the back side edge 12, and the crotch side edges 13A and 13B. The absorbent region 14 is configured by the aforementioned absorbent layer 21 being disposed (see FIGS. 3A and 3B). Both ends of the absorbent region 14 in the X-axis direction are disposed at positions separated from the crotch side edges 13A and 13B, respectively. An end of the absorbent region 14 on the positive side in the Y-axis direction is disposed at a position of the back side edge 12, and an end on the negative side in the Y-axis direction is disposed at a position of the abdominal side edge 11. However, the arrangement of the ends of the absorbent region 14 in the Y-axis direction is not specifically limited and the ends may not reach the positions of the abdominal side edge 11 and the back side edge 12.

The restraint portions 3A and 3B are members which are provided on the main body 2 along a pair of crotch side edges 13A and 13B, respectively, and suppressing leakage of the target object from the absorbent region 14. The restraint portions 3A and 3B may be formed of materials similar to materials of the backsheet 23. The restraint portions 3A and 3B are disposed extending in the Y-axis direction at positions spaced from each other and closer to the center line CL1 in the X-axis direction than the crotch side edges 13A and 13B, respectively. The restraint portions 3A and 3B are respectively fixed to the main body 2 at first edges 3a, 3a opposite the absorbent region 14, and second edges 3b, 3b on the absorbent region 14 side are configured to be openable to the absorbent region 14 (see FIG. 4).

The restraint portions 3A and 3B are fixed to the main body 2 on the first edges 3a, 3a sides, respectively. Specifically, among members constituting the restraint portions 3A and 3B, a portion to a crotch end from the first edges 3a, 3a is fixed to the main body 2 by a bonding method such as welding and adhesive. On the other hand, portions of the restraint portions 3A and 3B closer to the center line CL1 than the first edges 3a, 3a are not fixed to the main body 2. Thus, when worn, the second edges 3b, 3b of the restraint portions 3A and 3B lifts away from the main body 2. This allows the restraint portions 3A and 3B to open on the second edges 3b, 3b sides, respectively. Regions including edges 3c, 3c on the positive side and edges 3d, 3d on the negative side of the restraint portions 3A and 3B in the Y-axis direction are fixed to the main body 2. This forms, in the restraint portions 3A and 3B, fixing portions 26, 26 and fixing portions 27, 27 respectively that are fixed to the main body 2 at both ends in the Y-axis direction (longitudinal direction). In FIG. 2, the fixing portions 26, 26 and the fixing portions 27, 27 are hatched. Portions near the second edges 3b, 3b of the restraint portions 3A and 3B are manufactured to be stretchable in the Y-axis direction. Thus, the portions near the second edges 3b, 3b in the restraint portions 3A and 3B fit the legs near the wearer's crotch.

The first edges 3a, 3a of the restraint portions 3A, 3B are disposed at positions closer to the crotch side edges 13A and 13B than the absorbent region 14. The second edges 3b, 3b of the restraint portions 3A and 3B are disposed at positions closer to the center line CL1 than the ends of the absorbent region area 14 in the X-axis direction. However, a positional relationship between the restraint portions 3A and 3B and the absorbent region 14 is not specifically limited. The edges 3c, 3c of the restraint portions 3A and 3B on the positive side in the Y-axis direction are disposed at positions in the back side edge 12, and the edges 3d, 3d on the negative side in the Y-axis direction are disposed at positions in the abdominal side edge 11. The edges 3c, 3c of the restraint portions 3A and 3B are disposed closer directed to the positive side in the Y-axis direction than the absorbent region 14, and the edges 3d, 3d are disposed closer directed to the negative side in the Y-axis direction than the absorbent region 14. However, the positional relationship between the restraint portions 3A and 3B and the absorbent region 14 is not specifically limited.

The bonding member 4A is a strip-shaped member provided on the back side edge 12 side of the main body 2. The bonding member 4B is a strip-shaped member provided on the abdominal side edge 11 side of the main body 2. The bonding members 4A and 4B are constituted by a single sided tape having a bonding surface on one surface. The bonding members 4A and 4B are disposed to be stuck to a surface of the topsheet 22 of the main body 2 (see FIGS. 3A and 3B). The bonding members 4A and 4B are provided extending between the restraint portions 3A and 3B.

Both ends 4a and 4b of the bonding member 4A are disposed between the restraint portions 3A and 3B and the main body 2 on end sides of the restraint portions 3A and 3B on the positive side in the Y-axis direction. Both ends 4a and 4b of the bonding member 4B are disposed between the restraint portions 3A and 3B and the main body 2 on end sides of the restraint portions 3A and 3B on the negative side in the Y-axis direction. In other words, regions near the both ends 4a and 4b of the bonding members 4A and 4B are disposed to be inserted under inner surface sides of the restraint portions 3A and 3B. Inserted amounts of the bonding members 4A and 4B are not specifically limited, but it is preferable that the inserted amount is larger. For example, the ends 4a and 4b of the bonding members 4A and 4B are preferably disposed at positions closer to the first edges 3a, 3a in regions of the restraint portions 3A and 3B that are not fixed to the main body 2.

The bonding members 4A and 4B are provided on the main body 2 in a two-folded state (see FIGS. 3A, 3B and 4). The bonding members 4A and 4B in the two-folded state are configured so that the edges 4c, 4c on the absorbent region 14 side are openable to the absorbent region 14. Specifically, the edges 4d, 4d of the bonding members 4A and 4B opposite from the absorbent region 14, that is, opposite from the center line CL2, serve as fold lines. The edges 4c, 4c of the bonding members 4A and 4B include both edges 4e and 4f in a width direction of the bonding members 4A and 4B before being folded. At the edges 4c, 4c, the edges 4e and 4f are not fixed to each other and this allows to get close to and separate from each another.

The bonding member 4A is disposed at a position corresponding to the back side edge 12, and the bonding member 4B is disposed at a position corresponding to the abdominal side edge 11. The both ends 4a and 4b of the bonding members 4A and 4B are disposed between the restraint portions 3A and 3B and the main body 2 at positions adjacent to the fixing portions 26 and 27. Note that the positions adjacent to the fixing portions 26 and 27 may be positions where the restraint portions 3A and 3B come into contact with the fixing portions 26 and 27, or may be positions which are slightly separated from the fixing portions 26 and 27. The bonding members 4A and 4B are disposed along edges 14a and 14b of the absorbent region 14 which are opposite to each other in the Y-axis direction. Note that a state where the bonding members 4A and 4B are disposed along the edges 14a and 14b is a state where the bonding members 4A and 4B are disposed at positions near the edges 14a and 14b to extend in a direction substantially the same as the direction in which the edges 14a and 14b extend. In the present embodiment, the edges 14a and 14b extend along the X-axis direction, so that the bonding members 4A and 4B also extend along the X-axis direction. In the present embodiment, the bonding members 4A and 4B are disposed at positions near the edges 14a and 14b and overlapping the absorbent region 14. According to such a configuration, the target object is held back by the bonding members 4A and 4B before leaking out of the edges 14a and 14b. This allows the bonding members 4A and 4B to hold the target object within a range of the absorbent region 14. However, the bonding members 4A and 4B may be disposed at positions near the edges 14a and 14b and not overlapping the absorbent region 14, or the positions where only portions of the bonding members 4A and 4B overlap the absorbent region 14.

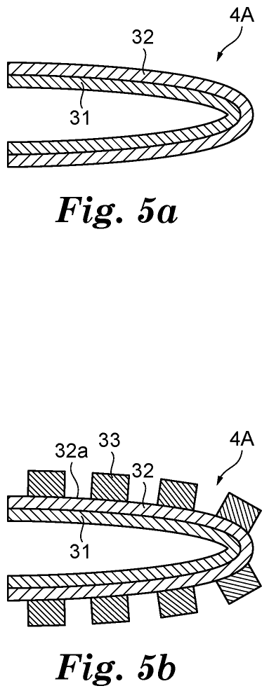

FIGS. 5A and 5B are enlarged cross-sectional view illustrating a layer configuration of the bonding member 4A. As illustrated in FIG. 5A, the bonding member 4A includes a base member 31 constituted by a sheet made of paper or the like, and a bonding layer 32 constituted by an adhesive or the like. In the two-folded state, the base member 31 is on a valley side, and the bonding layer 32 is on a mountain side. Accordingly, the bonding member 4A is bonded to the main body 2 by the bonding layer 32 being brought into contact with the main body 2. The bonding member 4A is bonded to the restraint portions 3A and 3B by the bonding layer 32 being brought into contact with the restraint portions 3A and 3B. When the diaper 1 is worn by the wearer, the bonding member 4A is stuck to the wearer by the bonding layer 32 being brought into contact with the wearer. Note that, as illustrated in FIG. 5B, a bonding surface 32a of the bonding member 4A may include a bonding suppressing member 33 that suppresses a bonding force of the bonding surface 32a. The bonding suppressing member 33 is a member not have a bonding force. The bonding suppressing member 33 is provided on a portion of the bonding surface 32a, and exposes another portion of the bonding surface 32a. This reduces an exposed area of the bonding surface 32a, and thus, the bonding force of the bonding surface 32a as a whole decreases. The bonding suppressing member 33 may be formed in a meshed shape or a dotted shape on the bonding surface 32a. Examples of a material for the bonding suppressing member 33 include a material constituting an absorbent article such as cotton, cellulose, rayon, polyolefin, and polyethylene terephthalate, and a material such as silicone and urethane. Alternatively, as the bonding suppressing member 33, a layer may be adopted that reduces the bonding force of the bonding surface 32a by being applied to an entire surface (or a portion of the surface) of the bonding surface 32a. Note that the bonding member 4B has a configuration having the same intent as the bonding member 4A.

The bonding members 4A and 4B are a portion that directly contacts the skin of the wearer. Therefore, a member providing low damage to the skin and low discomfort during wearing may be adopted as the bonding members 4A and 4B. For example, an adhesive used for the bonding members 4A and 4B may be an adhesive including a radiation cured silicone gel, and the silicone gel may include a cross-linked poly-diorganosiloxane material. The adhesive may be formed by exposing a composition including a poly-diorganosiloxane material to at least one of electron beam irradiation and gamma irradiation at a sufficient dose to cross-link the poly-diorganosiloxane material. The poly-diorganosiloxane material may also include polydimethylsiloxane. Polydimethylsiloxane may be selected from the group consisting of one or more silanol terminated polydimethylsiloxanes, one or more non-functional polydimethylsiloxanes, and combinations thereof. The polydimethylsiloxane may consist of one or more non-functional polydimethylsiloxanes. The adhesive may further include a silicate resin tackifier. The adhesive may further include a poly(dimethylsiloxane-oxamide) linear copolymer.

The poly-diorganosiloxane material includes a poly-diorganosiloxane fluid having a dynamic viscosity at 25° C. of no greater than 1000000 mPa·s sec. The poly-diorganosiloxane material may consist of poly-diorganosiloxane fluids having a kinematic viscosity at 25° C. of no greater than 100000 centistokes. The adhesive may have a 180 degree peel adhesion strength from human skin of no greater than 200 grams per 2.54 centimeters as measured according to the Skin Peel Adhesion Procedure. For example, an adhesive applied to the bonding members 4A and 4B may include a terminal silanol functional PDMS fluid, a MQ tackifier, and a thickener. As an example, the adhesive contains 87 parts by weight of a terminal silanol functional PDMS fluid and 13 parts by weight of a MQ tackifier, per 100 parts by weight thereof. The adhesive may contain 10 parts by weight of a thickener per 100 parts of a PDMS fluid.

Next, the operation and effect of the diaper 1 according to the present embodiment will be described.

The diaper 1 includes strip-shaped bonding members 4A and 4B provided on the abdominal side edge 11 and the back side edge 12 of the main body 2. The bonding members 4A and 4B are provided extending between a pair of restraint portions 3A and 3B. With such a configuration, the bonding members 4A and 4B are bonded to the portion on the abdominal side and the portion on the back side of the wearer when the diaper 1 is worn by the wearer. This allows the portion between the pair of restraint portions 3A and 3B of the main body 2 to be fixed to a portion of the body of the wearer via the bonding members 4A and 4B. This can suppress the leakage of the target object from the main body 2 at the location where the bonding members 4A and 4B are provided. In a case that the second edges 3b, 3b of the restraint portions 3A and 3B on the absorbent region 14 side are configured to be openable to the absorbent region 14, and the second edges 3b, 3b, which are free ends, are inclined toward the CL1 side, the both ends of the bonding members 4A and 4B are disposed between the restraint portions 3A and 3B and the main body 2 on one end side in the longitudinal direction of these. According to such a configuration, when the bonding members 4A and 4B are bonded to the wearer, and thereby, are pulled toward the wearer, one end side of each of the restraint portions 3A and 3B in the longitudinal direction rises toward the wearer side with the bonding members 4A and 4B (see, FIG. 4, for example). One end side of each of the restraint portions 3A and 3B in the longitudinal direction is a portion that is unlikely to rise, and thereby, is prone to generate a gap until the body of the wearer, but since each of the bonding members 4A and 4B can raise the relevant portion, the gap until the wearer can be reduced. This allows the function of suppressing the leakage from the diaper 1 to be improved.

The bonding members 4A and 4B are provided on the main body 2 in a two-folded state, and the edges 4c, 4c on the absorbent region 14 side are configured to be openable to the absorbent region 14. According to such a configuration, the bonding members 4A and 4B can capture the target object in a space that opens to the absorbent region 14 side. The edges 4c, 4c of the bonding members 4A and 4B opening to the absorbent region 14 side can raise the restraint portions 3A and 3B high.

The restraint portions 3A and 3B include the fixing portions 26 and 27 each of which is fixed to the main body 2 on one end side in the longitudinal direction, and the both ends 4a and 4b of the bonding members 4A and 4B are disposed between the restraint portions 3A and 3B and the main body 2 at the positions adjacent to the fixing portions 26 and 27. The fixing portions 26 and 27 are portions where the restraint portions 3A and 3B are unlikely to rise. The bonding members 4A and 4B being disposed at the positions described above allows the portions near the fixing portions 26 and 27 to rise.

The bonding members 4A and 4B may be disposed along the edges 14a and 14b of the absorbent region 14. According to such a configuration, the bonding member can hold back the target object at a portion near the edge of the absorbent region. Such a configuration can more broadly use the absorbent region 14 as compared with a configuration in which the bonding members 4A and 4B are farther away from the edges 14a and 14b toward the center line CL2. Furthermore, such a configuration can suppress the target object overflowing outside the absorbent region 14 as compared with a configuration in which the bonding members 4A and 4B are farther away from the edges 14a and 14b toward outside the absorbent region 14.

The bonding surface 32a of each of the bonding members 4A and 4B includes the bonding suppressing member 33 that suppresses the bonding force of the bonding surface 32a. According to such a configuration, the bonding force of the bonding surface 32a with respect to the wearer can be appropriately adjusted. Suppressing the bonding force of the bonding surface 32a facilitates positioning when the bonding members 4A and 4B are provided on the main body 2.

The present invention is not intended to be limited to the embodiment described above.

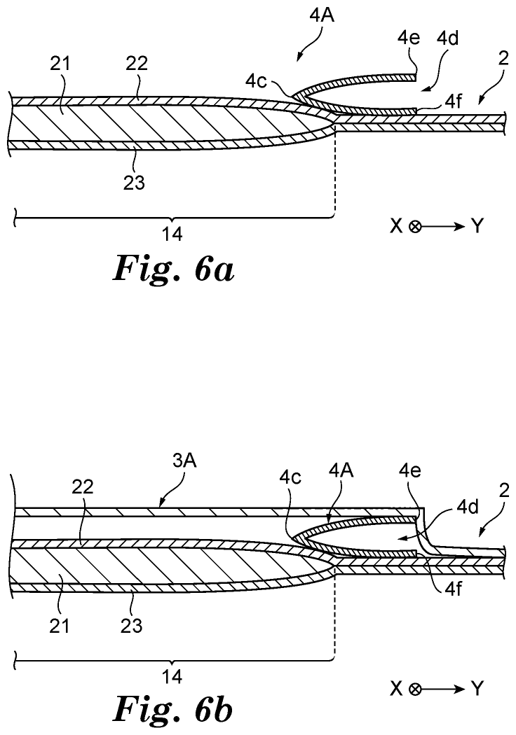

For example, a configuration illustrated in FIGS. 6A and 6B may be adopted. In the diaper illustrated in FIG. 6, the bonding member 4A is provided to be openable to an outside of the absorbent region 14. Specifically, the edge 4c of the bonding member 4A on the absorbent region 14 side serves as a fold line, and the opposite edge 4d includes the both edges 4e and 4f in the width direction of the bonding member 4A.

In the embodiment described above, although both the bonding member 4A on the back side and the bonding member 4B on the abdominal side are provided, only one of the bonding members 4A and 4B may be provided, and the other may be omitted. The bonding members 4A and 4B are respectively provided on only one location on the back side and the abdominal side, but may be provided in a plurality of locations.

The embodiment described above illustrates, as an absorbent article, the open-type diaper 1 in which the abdominal side edge 11 and the back side edge 12 can be attached and detached via the fastener members 16A and 16B. Instead of this configuration, a pant-type or pad type diaper may be adopted as the absorbent article. The absorbent article may also be a napkin, a urine leakage pad, or the like, not limited to a diaper. The target object may also be a body fluid such as menstrual blood, vaginal discharge, and sweat.

In the embodiment described above, the pair of restraint portions 3A and 3B are respectively fixed to the main body 2 on the first edges 3a, 3a side opposite the absorbent region 14, and second edges 3b, 3b on the absorbent region 14 side are configured to be openable to the absorbent region 14. Instead of this configuration, the pair of restraint portions 3A and 3B may have the first edges 3a, 3a opposite the absorbent region 14 that are configured to be openable. In such a configuration, the second edges 3b, 3b sides are fixed to the main body 2. Accordingly, even when the bonding members 4A and 4B are pulled toward the wearer because of being bonded to the wearer, such an effect is not obtained that one end side of each of the restraint portions 3A and 3B in the longitudinal direction rises toward the wearer side with the bonding members 4A and 4B. However, since the bonding members 4A and 4B are provided across an entire region between the pair of restraint portions 3A and 3B, the function of suppressing the leakage can be exhibited when the bonding members 4A and 4B are bonded to the wearer. Note that in such a configuration, the strip-shaped member constituting the restraint portions 3A and 3B is two-folded in the width direction, one of the pieces may be fixed to the topsheet 22, and the other of the pieces may not be fixed to the main body 2. In such a configuration, the other piece that is not fixed to the main body 2 functions as each of the restraint portions 3A and 3B. In such a configuration, in the fixing portions 26 and 27, each of the restraint portions 3A and 3B is fixed to the piece itself fixed to the topsheet 22 rather than to the topsheet 22. As described above, in the fixing portions 26 and 27 of the restraint portions 3A and 3B, the portion that rises as the restraint portion 3A or 3B may not be directly fixed to the main body 2, or may be fixed via the piece fixed to the main body 2.[0001]

In the above-described embodiment, the restraint portions 3A and 3B are configured as the separate members from the main body 2. Instead of this configuration, the restraint portions 3A and 3B may be integrally formed with the member constituting the main body 2. For example, the backsheet 23 and the topsheet 22 may be extended such that the members of the back sheet 23 and the top sheet 22 may constitute the restraint portions 3A and 3B.

### Embodiments

The absorbent article according to an aspect of the present invention includes a bonding member in a strip shape provided to the main body on a side of at least one of the abdominal side edge and the back side edge. The bonding member is provided extending between the pair of restraint portions. With such a configuration, the bonding member is bonded to at least one of a portion on the abdominal side and a portion on the back side of the wearer when the absorbent article is worn by the wearer. This allows the portion between the pair of restraint portions of the main body to be fixed to a portion of the body of the wearer via the bonding member. This can suppress the leakage of the target object from the main body at the location where the bonding member is provided. Both ends of the bonding member are disposed between the restraint portions and the main body at one end side of the restraint portion in a longitudinal direction. Among such a configuration, in a configuration that the pair of restraint portions are fixed to the main body to be closer to the crotch side edge than the first edge opposite the absorbent region, and the second edge on the absorbent region side is configured to be openable to the absorbent region, when the bonding member is bonded to the wearer, and thereby, are pulled toward the wearer, one end side of the restraint portion in the longitudinal direction rises toward the wearer side with the bonding member. One end side of each of the restraint portions in the longitudinal direction is a portion that is unlikely to rise, and thereby, is prone to generate a gap until the body of the wearer, but since the bonding member can raise the relevant portion, the gap until the wearer can be reduced. This allows the function of suppressing the leakage from the absorbent article to be improved.

The bonding member may be provided on the main body in a two-folded state, and may include an edge on an absorbent region side configured to be openable to the absorbent region.

The restraint portion may include a fixing portion fixed to the main body at one end side in the longitudinal direction, and the both ends of the bonding member may be disposed between the restraint portions and the main body at positions adjacent to the fixing portion.

The bonding member may be disposed along an edge of the absorbent region.

A bonding surface of the bonding member may include a bonding suppressing portion configured to suppress a bonding force of the bonding surface.

1. An absorbent article covering a crotch portion of a wearer from an abdominal side to a back side with a sheet-like main body, the absorbent article comprising:
the main body including an abdominal side edge, a back side edge, a pair of crotch side edges extending between the abdominal side edge and the back side edge, and an absorbent region in which an absorbent is disposed;
a pair of restraint portions provided on the main body along the pair of crotch side edges, and configured to suppress leakage of a target object from the absorbent region; and
a bonding member in a strip shape provided to the main body on a side of at least one of the abdominal side edge and the back side edge, wherein
the bonding member is provided extending between the pair of restraint portions, and
both ends of the bonding member are disposed between the restraint portions and the main body at one end side of the restraint portion in a longitudinal direction.

2. The absorbent article according to claim 1, wherein the bonding member is provided on the main body in a two-folded state, and includes an edge on an absorbent region side configured to be openable to the absorbent region.

3. The absorbent article according to claim 1, wherein the restraint portion includes a fixing portion fixed to the main body at one end side in the longitudinal direction, and the both ends of the bonding member are disposed between the restraint portions and the main body at positions adjacent to the fixing portion.

4. The absorbent article according to claim 1, wherein the bonding member is disposed along an edge of the absorbent region.

5. The absorbent article according to claim 1 4, wherein a bonding surface of the bonding member includes a bonding suppressing member configured to suppress a bonding force of the bonding surface.
